(19) United States
(12) Patent Application Publication
(10) Pub. No.: US 2017/0367684 A1
(43) Pub. Date: Dec. 28, 2017

(54) SYSTEMS AND METHODS FOR SUPER-RESOLUTION COMPACT ULTRASOUND IMAGING

(71) Applicant: INNOMIND TECHNOLOGY CORPORATION, Richmond Hill, Ontario (CA)

(72) Inventor: Foroohar FOROOZAN, Richmond Hill (CA)

(21) Appl. No.: 15/532,191

(22) PCT Filed: Jan. 5, 2016
(86) PCT No.: PCT/CA2016/050006

§ 371 Date: Jun. 1, 2017

Related U.S. Application Data
(60) Provisional application No. 62/099,680, filed on Jan. 5, 2015.

(51) Int. Cl. A61B 8/08; G01S 15/89; G01S 7/52

(52) U.S. Cl. CPC A61B 8/5207; G01S 7/52047; G01S 15/8977

(57) ABSTRACT

Systems and methods for medical imaging, specifically ultrasound imaging capable of achieving spatial resolutions that can resolve point objects smaller than 100 μm irrespective of them to be well-resolved, using the principles of compressive sensing and sparse recovery are described. Ultrasound system uses the transmit transducers sequentially to sonicate the medium and the data is acquired over the receive transducers. The acquired signals are then sampled by the low-dimensional acquisition system. The signals are recovered using an optimization method before a frequency domain beamforming technique is applied. The time reversal focused frequency matrix is formed to focus the energy of different frequency bands into a single frequency. Next, a super-resolution synthetic time reversal Phase Coherent MUltiple SIgnal Classification (PC-MUSIC) method is applied to focus spatially on the target locations considering the frequency dependent phase response of the transducers and the green's function of the ROI at the focused frequency.

### CROSS REFERENCE TO RELATED APPLICATION

The present application claims the benefit of U.S. provisional patent application No. 62/099,680 filed on Jan. 5, 2015 and entitled SYSTEMS AND METHODS FOR SUPER-RESOLUTION COMPACT ULTRASOUND IMAGING, the entire contents of which are incorporated herein by reference.

### FIELD OF INVENTION

The present disclosure relates to systems and methods for medical imaging and, in particular, to ultrasound imaging. Certain examples of the disclosure provide systems and methods for super-resolution compressed ultrasound imaging capable of micrometer resolutions. This disclosure comprises of systems and methods for (i) acquisition; and (ii) processing of ultrasound imaging data.

### BACKGROUND

Ultrasound is an imaging modality that is relatively cheap, risk-free, radiation-free and portable.

However, in some applications, the resolution of ultrasound images is very low, limiting the application of this imaging modality. For example, ultrasound brain vascular imaging has not been clinically achieved due to spatial resolution limitation in ultrasound propagation through the human skull; this limits the application of ultrasound in Traumatic Brain Injury (TBI) for emergency situations. Another example is breast cancer screening where ultrasound is not solely and frequently used for population-based screening of the breast cancer due to ultrasound-limited resolution.

The second problem with ultrasound is that in some applications, there is a need to use a large number of transducers (sometimes as high as a couple of thousands) producing several hundreds of frame rate per second and each frame has several of hundreds of image lines. Therefore, the processing power is high in current ultrasound machines to be able to process a large amount of data in real-time. In order to use ultrasound in emergency and point-of-care applications, the imaging system should be compact with lower acquisition and processing requirements.

Therefore, there are two aspects in improving the performance of current ultrasound systems (i) to improve the image quality not by increasing the quantity of the acquired data; and (ii) to accelerate the acquisition and processing rates and at the same time not dropping the quality in terms of image resolution, Signal-to-Noise ratios (SNRs), and contrast.

Compressive sensing (CS) approaches provide an alternative to the classical Nyquist sampling framework and enable signal reconstruction at lower sampling rates, for example by Candes et. al., in “Robust uncertainty principles: exact signal reconstruction from highly incomplete frequency information,” IEEE Transactions on Information Theory, vol. 52, no. 2, pp. 489-509, February 2006. The idea of CS is to merge the compression and sampling steps. In recent years, the area of CS has branched out to a number of new applications like radar, communications, and ultrasound imaging.

All the proposed CS approaches in ultrasound imaging is using a non-adaptive beamforming (“spatial filtering”) to reconstruct the final image in ultrasound. This non-adaptive beamforming in based on a Delay-and-Sum (DAS), which is a preferred beamforming method in current ultrasound machines. In the DAS approach, relevant time-of-flights from each transducer element to each point in the region of interest (ROI) are compensated and then a summation is performed on all the aligned observations to form the image. The DAS beamformer is independent of data with fixed weights and in order to apply this techniques in time domain, the data samples should be high enough even more than the rate dictated by the Shannon-Nyquist theorem. Now, combining DAS with CS provides lower resolution as compared to applying super resolution techniques like Time Reversal MUltiple SIgnal Classification (TR-MUSIC) and Capon methods.

The time reversal (TR)-based imaging methods utilize the reciprocity of wave propagation in a time-invariant medium to localize an object with higher resolution. The focusing quality in the time-reversal method is decided by the size of the effective aperture of transmitter-receiver array. This effective aperture includes the physical size of the array and the effect of the environment. A complicated background will create the so-called multipath effect and can significantly increase the effective aperture size, which enhances the resolution of the acquired images.

Most of the previous computational time reversal based imaging methods uses the eigenstructure of the TR matrix to image the targets. Generally, the singular value decomposition (SVD) of the TR matrix is needed for every frequency bin and for every space-space TR-matrix. For ultrawideband (UWB) imaging, the SVDs of space-space TR matrices are utilized and combined to form the final image. There are two problems with this configuration: (i) the computational complexity of repeating the SVD of the TR matrix in every frequency bin is very high limiting the usage of this technique in real-time ultrasound system and (ii) at each frequency, the singular vectors have an arbitrary and frequency-dependent phase resulted from the SVD.

In UWB TR_MUSIC method, only the magnitude of the inner products are combined along the bandwidth and these arbitrary phases cancel out, therefore, the problem of incoherency does not exist for non-noisy data. However, the super-resolution property of TR-MUSIC disappears as the signals become noisy which is due to the random phase structure induced by noise. A modified version of TR-MUSIC, Phase Coherent MUSIC (PC-MUSIC) uses a re-formulation of TR-MUSIC, which retains the phase information and also applies averaging of the pseudospectrum in frequency to cancel out the random phase degradation of TR-MUSIC in case of noisy data. The problem with PC-MUSIC is that since it uses phase information and disregards the phase response of the transducers, its ability to localize the targets at their true locations is adversely impacted as explained in “Super-resolution ultrasound imaging using a phase-coherent MUSIC method with compensation for the phase response of transducer elements,” IEEE Transactions on Ultrasonics, Ferroelectrics, and Frequency Control, vol. 60, no. 6, pp. 1048-1060, June 2013.

A modification to PC-MUSIC was proposed by Labyed et al. to compensate the transducer phase response by developing an experimental method to estimate the phase responses beforehand. The computational complexity of this modification is still high as the SVD is needed for every frequency bin across the bandwidth and the image is formed by averaging these pseudospectrums for points in the region-of-interest (ROI). Also, the efficiency of this incoherent approach depends on the SNRs of the individual frequency bins.

Frequency matrices were proposed previously by Kaveh et al. in “Focusing matrices for coherent signal-subspace processing,” IEEE Transactions on Acoustics, Speech and Signal Processing, vol. 36, no. 8, pp. 1272-1281, August 1988, for finding the direction-of-arrival of multiple wideband sources using passive arrays. Li et. al modified these matrices to be used in active arrays with robust Capon beamformers in ultrasound imaging.

### BRIEF SUMMARY OF THE INVENTION

An embodiment of the present invention that is described herein provides a method comprising of sending ultrasound plane wave to a ROI comprising of multiple point scatterers form the transducer elements of the array sequentially, a low-dimensional data acquisition method to receive the backscatters from the medium by all the transducer elements and a super-resolution image reconstruction method to form the final image of the ROI irrespective of the sparsity of the received signals.

In disclosed embodiment, the low-dimensional acquisition method is based on the principle of compressive sensing and sparse recovery. By way of example, the sensing matrices are based on random Gaussian matrices and the recovery is based on Fourier transform or wave atom of the received data channel. The reader is referred to the following publication that is hereby expressly incorporated by reference and is written by the current writer of this patent application: “Wave Atom Based Compressive Sensing and Adaptive Beamforming for Ultrasound Imaging”, IEEE ICASSP 2015, PP. 2474-2478.

By way of example, sub-Nyquist sampling schemes that can be used in the low-dimensional sampling by unit 303 are described by Gedalyahu et al., in “Multichannel Sampling of Pulse Streams at the Rate of Innovation,” IEEE Transactions on Signal Processing, volume 59, number 4, pages 1491-1504, 2011, which is incorporated herein by reference. Example hardware that can be used for this purpose is described by Baransky et al., in “A Sub-Nyquist Radar Prototype: Hardware and Algorithms,” IEEE Transactions on Aerospace and Electronics Systems, pages 809-822, April 2014, which is incorporated herein by reference.

In another embodiment, the recovered signals in frequency are used to form the full data matrix. The beamforming uses focused frequency time reversal (FFTR) matrices to focus in frequency for UWB ultrasound signals, as well as time reversal Phase Coherent MUltiple Signal Classification (PC-MUSIC) algorithm to focus spatially on the target location. This combined method, which is referred to as FFTR-PCMUSIC, is motivated by the pressing need to improve the resolution of diagnostic ultrasound systems. Compared with the TR matched filter (TRMF) and incoherent TR-MUSIC approaches, the method proposed in this disclosure has lower computational complexity, higher visibility, higher robustness against noise, and higher accuracy for imaging point targets when the targets are micrometer distance apart. The reader is referred to the following publication that is hereby expressly incorporated by reference and is written by the current writer of this patent application: “Super-resolution Ultrawideband Ultrasound Imaging using Focused Frequency Time Reversal MUSIC”, IEEE ICASSP, 2015, 887-891.

The FFTR-PCMUSIC uses the TR focusing in time and space to achieve high temporal and spatial resolution. The background Green's function at the focused frequency is used as the steering vector to form the final image. This method reduces the effect of noise on target localization accuracy as well as the computational complexity needed for subspace-based methods for UWB ultrasound data by using frequency-focusing matrices together with the focused frequency Green's function. Effectively, the maximum resolution achieved by the FFTR-PCMUSIC is inherently limited by the SNR and the bandwidth of the transducers.

### BRIEF DESCRIPTION OF THE DRAWINGS

FIG. 1 is a flowchart setting forth the steps of the proposed method for compact acquisition and reconstruction of a high-resolution image in an ultrasound system.

FIG. 2 is a block diagram of an example of an ultrasound system using this method.

FIG. 3 shows the hardware of the system using the functional diagrams presented in FIGS. 1 and 2.

FIG. 4 shows the signal path of an example transmit-receive path from each transmitter transducer to M receiver transducers considered in accordance with an embodiment of the present invention. This path is repeated for each transmitter in the array.

FIG. 5 shows the geometry of a 2D array of transducer with 2D ROI, in accordance with an embodiment of the present invention.

FIG. 6, by way of example, shows a simulation of the ROI with 2, 3, and 10 point targets and the results from applying the method presented in this disclosure.

FIG. 7, by way of example, shows a real ultrasound data from a wire phantom and point targets after applying the method presented in some of the embodiments of this invention.

### DETAILED DESCRIPTION OF INVENTION

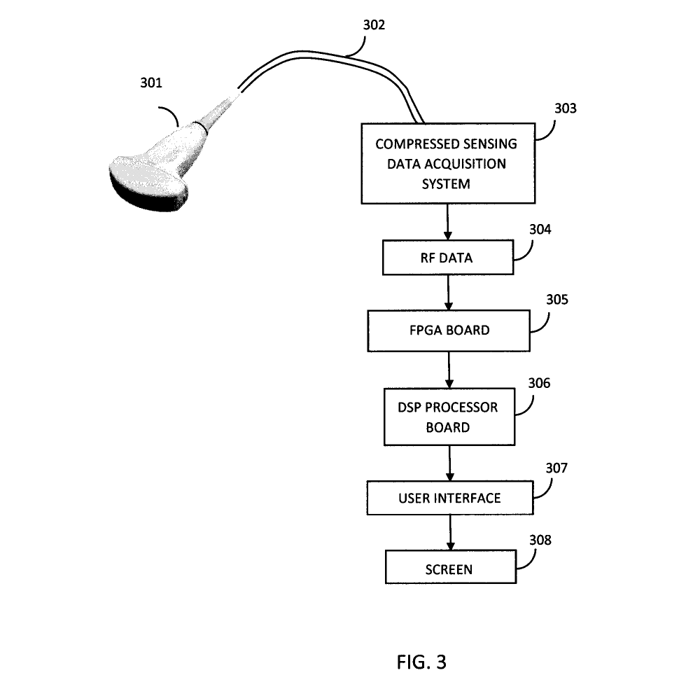

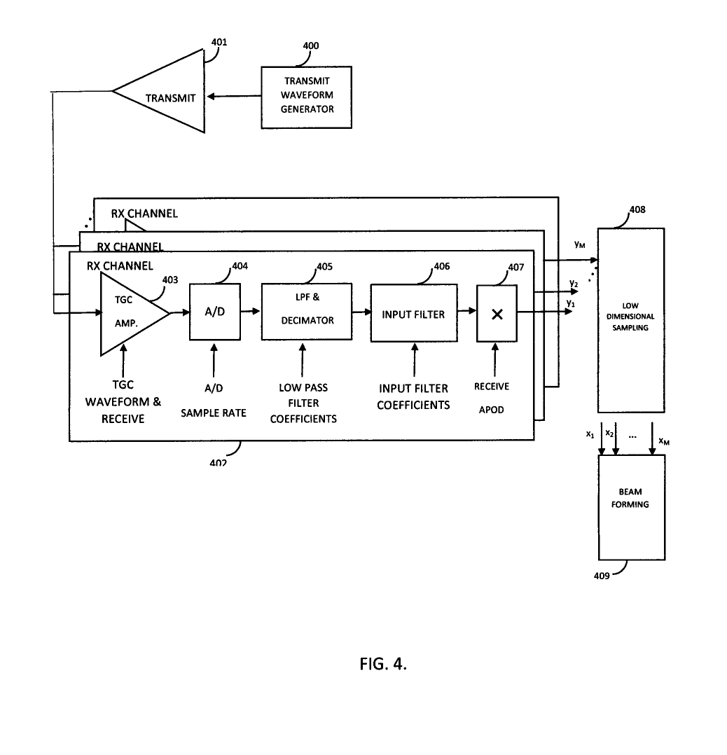

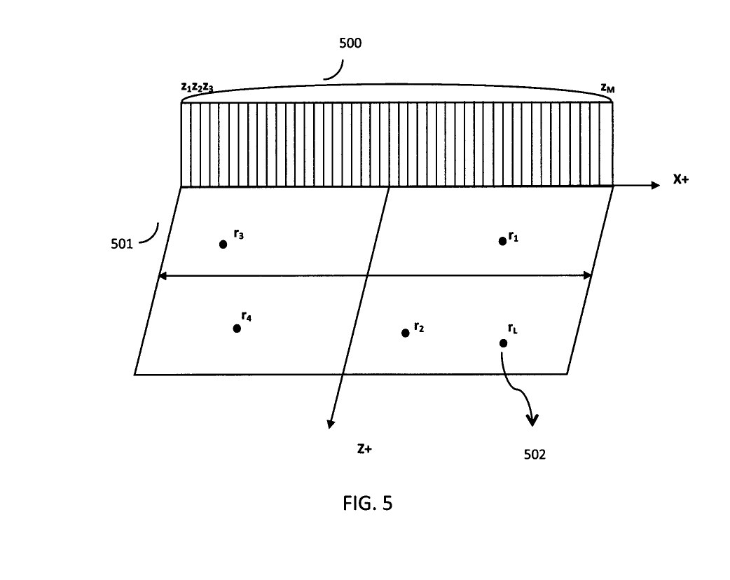

The transducer array (M transducers) shown in FIG. 3 as “301” sends a short pulse generated by way of example from the transmit waveform (FIG. 4, “400”) sequentially from each transducer to the medium. The medium comprises of point scatterers as shown in FIG. 5, “502” embedded in a medium speckle noise. The data signals are recorded through the received circuitry as shown in FIG. 4, “402” using the receive transducer array (units “301” or “500”).

All the transducers in the array are sending a plane wave one by one and the same transducer array receives and records the backscatters from the medium. As shown in FIG. 5, “502”, the point scatterers are located at rₗ in the ROI. Due to a probing signal fⱼ(t) sonicated by the transducer j, a pressure filed is generated at the location of the scatterer as qⱼ(rₗ, t)=qⱼ(t)δ(rₗ), where δ(rₗ) is delta function at point rₗ with strength qⱼ(t) which depends on the probing signal fⱼ(r), the attenuation of the medium in forward direction, the electromechanical impulse response of the transmit transducer. By way of example, in frequency domain, the field generated at the scatterer location is Qⱼ(rₗ, ω).

The Green's function of the medium is the spatio-temporal impulse response of the medium shown as “501” in FIG. 5. By way of example, in frequency domain the integral of the medium Green's function over the surface of the transducer, is given as following.

$$G ( {z}_{i} , {r}_{l} , ω ) = \int {\int }_{{S}_{i}} \frac{{e}^{- i \overline{k}} \vert {r}_{l} - {z}_{i} \vert}{4 π \vert {r}_{l} - {z}_{i} \vert} dS , \qquad ( 1 )$$

where zᵢ is the location of the transducer i array as shown as unit “500” in FIG. 5, and

$$\overline{k} = \frac{ω}{c} - i α ,$$

with c being the sound propagation speed, and α is the amplitude of the attenuation coefficient of the environment, see “Super-resolution ultrasound imaging using a phase-coherent MUSIC method with compensation or the phase response of transducer elements,” IEEE Transactions on Ultrasonics, Ferroelectrics, and Frequency Control, vol. 60, no. 6, pp. 1048-1060, June 2013.

The pressure filed at the received transducer location i is

yᵢⱼ(ω)=Hᵢⱼ(ω)Qⱼ(rₗ,ω)G(zᵢ,rₗ,ω)+vᵢⱼ(ω) (2),

where Hᵢⱼ(ω) is the forward-backward frequency response of the transducers i and j, and vᵢⱼ(ω) is the measurement noise.

The signals yᵢⱼ(ω) is filtered and sparsified in the frequency domain by way of example using a wavelet de-noising tool as shown in FIG. 4, unit “406”.

The filtered signal yᵢⱼ(ω) is down-sampled (“102”) to 1/k'th of the original samples using the random sensing matrices φ, reducing the sampling matrix size to K×M, with K<<N as follows:

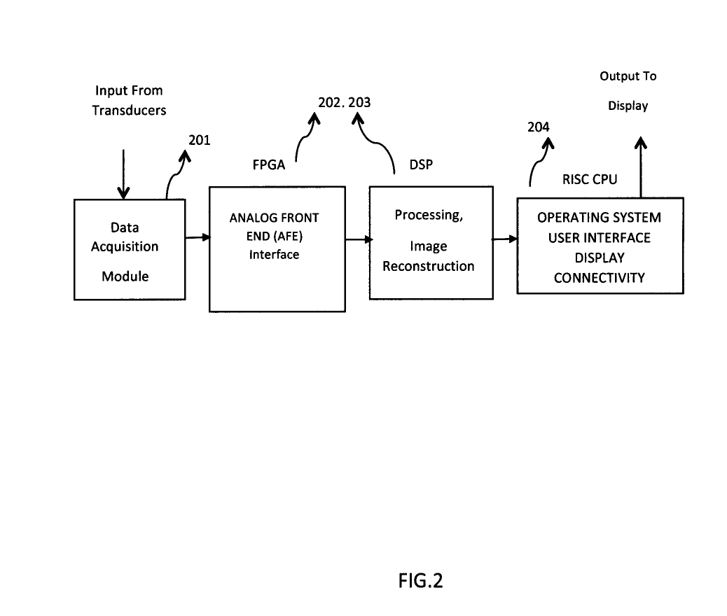

xᵢⱼ=φyᵢⱼ+e (3)

where xᵢⱼ is the down-sampled data at transducer i and e is the measurement error. This phase is just to get the down-sampled data and in practice, this stage is the output of the modified data acquisition system of an ultrasound system shown in FIG. 2 as “201”. This modified data acquisition system is called low-dimensional acquisition system in this disclosure.

In recovery, a regularized-l1 optimization is used to find the sparsest solution of yᵢⱼ by way of example as the wave atom basis or Fourier basis. The optimization problem is

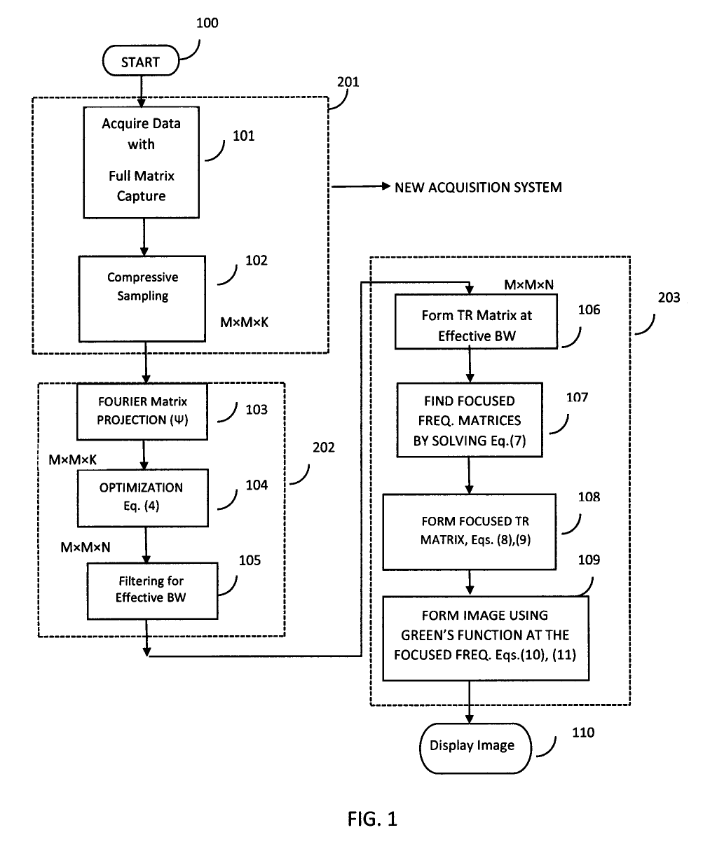

½∥φyᵢⱼ−xᵢⱼ∥₂+τ∥Ψyᵢⱼ+e (4)

where Ψ is the wave atom or Fourier dictionary, τ is a regularization parameter, and ∥.∥₂, ∥.∥₁ are l₂- and l₁-norms of the vectors. The minimization formula in (4) finds the signals yᵢⱼ. This step is shown in FIG. 1 as 103, 104 and 202 in FIG. 2. In various embodiments, unit 104 may solve the optimization problem of Equation (4) in any suitable way. Example optimization schemes that can be used for this purpose are second-order methods such as interior-point methods described by Candes and Romberg, in “11-magic: Recovery of Sparse Signals via Convex Programming,” October, 2005; and by Grant and Boyd, in “The CVX User's Guide,” CVX Research, Inc., November, 2013; and YALL1 basic models and tests by J. Yang and Y. Zhang. “Alternating direction algorithms for L1-problems in compressive sensing”, SIAM Journal on Scientific Computing, 33, 1-2, 250-278, 2011, which are incorporated herein by reference.

The signals yᵢⱼ are filtered to increase the SNR before going to the beamforming process as shown in unit 105.

In practice, the step in [0031] is not needed and it is directly acquired at the modified data acquisition of the ultrasound system shown in FIG. 2, 201. Here, it is performed offline for the sake of conceptual clarity.

After recovery of signals, to beamform the M signals for image reconstruction, the FFTR-PCMUSIC method is used as shown in FIG. 4., “409”. This method uses TR focusing frequency matrices to focus on frequency first and then uses the focused frequency TR matrix and a modified MUltiple SIgnal Classification (MUSIC) algorithm to focus spatially on the target location as shown in blocks 106-109 in FIG. 1.

This method uses the TR-PCMUSIC in conjunction with TR-based frequency focusing matrices to reduce the computational complexity of incoherent TR-MUSIC as well as phase ambiguity of the PCMUSIC in a noisy ultrasound environment. In FFTR-PCMUSIC, the SVD is applied once into a focused frequency TR matrix through finding unitary focusing matrices and applying a weighted averaging of the focused TR matrix over the bandwidth. This averaging reduces the effect of noise in space-space FFTR-PCMUSIC since the signal subspace is used after focusing in frequency. Also, after forming the FFTR matrix, the signal and noise subspaces are used once in forming the pseudo-spectrum which peaks at the locations of the point targets.

In step 100291 we have the reconstructed signal \tilde{y}ₘ, denoting Q as the frequency band of interest after signal sparsifying in frequency domain, and ω_{q} being the frequency of each band. Then, we have Q of M×M space-space matrices K(ω_{q}) as follows.

K(ω_{q})=F(ω_{q})Σₗ₌₁^{L}τₗg(ω_{q},rₗ)g^{T}(ω_{q},rₗ)+v(ω_{q}) (5)

where L is the number of scatterers shown in FIG. 5 as “502”, and the green's vector

g(ω_{q},rₗ)=e^{iφ(ω}^{_{q}}⁾[G(z₁,rₗ,ω), . . . ,G(z_{M},rₗ,ω)]̂T (6),

F(ω_{q}) takes care of both the field generated at the source location Qⱼ(rₗ,ω) and the frequency response of the transducers, assuming all to be the same. The frequency dependent phase of the transducer is denoted as φ(ω_{q}).

In practice, the transducer phase response can be calculated by experimenting on a single point target embedded at a known location of a homogeneous environment, as demonstrated in “Super-resolution ultrasound imaging using a phase-coherent MUSIC method with compensation or the phase response of transducer elements,” IEEE Transactions on Ultrasonics, Ferroelectrics, and Frequency Control, vol. 60, no. 6, page. 1048-1060, June 2013.

The TR matrix T(ω_{q})=K(ω_{q})^{H}K(ω_{q}) is computed at every frequency bin. In order to find the focused frequency TR matrix \tilde{T}(ω₀), I am using the unitary matrices B(ω_{q}) to minimize the difference between T(ω₀) and the transformed TR matrix at frequency q with the following minimization problem.

min ∥K(ω₀)^{H}−B(ω_{q})K(ω_{q})^{H}∥_{F} (7)
Subject to B(ω_{q})^{H}B(ω_{q})=I,
   where ∥.∥I_{F} is the Frobenious norm. The solution to this problem is given as

B(ω_{q})=V(ω_{q})U(ω_{q})^{H}, (8)

where V(ω_{q}) and U(ω_{q}) are the right and left singular vectors of the TR matrix K(ω_{q})^{H}K(ω₀). Then, the coherently focused TR operator is the weighted average of the transformed matrix of TR with unitary matrix B(ω_{q}) as follows.

\tilde{T}(ω₀)=Σ_{q=0}^{Q-1}β_{q}B(ω_{q})T(ω_{q})B(ω_{q})^{H} (9)

where β_{q} is the weight proportional to the SNR of q'th bin. These steps are shown in FIG. 1 as “107” and “108”.

The advantage with this approach is that the Green's function at the focused frequency is used for image formation. It is worth noting that for incoherent TR-MUSIC and PC-MUSIC, the array steering vector should be computed for every frequency bin over the entire grid, which is computationally expensive.

The final step will be to form the pseudo-spectrum of the FFTR-PCMUSIC as follows.

$$A ( {ω}_{0} , r ) = {e}^{- i φ ( {ω}_{0} )} \frac{{g}^{H} ( {ω}_{0} , r ) \tilde{U} ( {ω}_{0} , r ) {\tilde{V}}^{H} ( {ω}_{0} , r ) g ( {ω}_{0} , r )}{{‖ g ( {ω}_{0} , r ) ‖}^{2}} \qquad ( 10 )$$

where Ũ(ω₀, r) and \tilde{V}(ω₀, r) are the left and right singular matrices at the focused frequency resulted from the SVD of \tilde{T}(ω₀), g(ω₀, r) is the background green's function at the focused frequency and observation point r in the ROI. (Refer to unit “109” in FIG. 1).

As shown in FIG. 1. (“109”), the FFTR-PCMUSIC image is given by

$$I ( r ) = \frac{1}{1 - A ( {ω}_{0} , r )}$$

which peaks at the location of scatterers with high resolution.

FIG. 2 shows the functional block diagram of the ultrasound system using the above methods. The acquisition system is a low dimensional data acquisition system (module 201) and a field-programmable gate array (FPGA) board 202 is responsible for the connection to the beamformer. A Digital Signal Processing (DSP) board (203) can be used in which the recovery of signals based on modules 103-105 is be implemented. The FFTR-PCMUSIC beamforming based on modules 106-110 is implemented in the DSP board as well to reconstructing the final image.

By way of example, FIG. 3 presents system modules that use the methods for high-resolution compressed ultrasound imaging. The system comprises of a transducer array, which excites the ROI and receives the backscatters from the medium.

The system of FIG. 3 further comprises of compressed sensing data acquisition module (303), which records the signals received by the transducers using a low-dimensional sampling method.

The digital rf data acquired in module 304 of FIG. 3, is further processed by an FPGA module (305) which provides a connection from the low-dimensional acquisition module to the DSP board of 306.

The DSP board comprises of a programming executable in the processor to recover the full capture matrix from the sparse data acquired by the low-dimensional acquisition module.

The DSP board comprises of a programming executable in the processor to reconstruct the image of the ROI using the FFTR-PCMUSIC method.

The user interface module in FIG. 3. (307) comprises of a connection between the DSP board and the screen of module 308 to display the image.

The signal path presented in FIG. 4 is an example based on Verasonics ultrasound system and it is purely chosen for the sake of clarity. The transmit transducers fires plane acoustic wave sequentially from all M elements. The low-dimensional sampling unit 408, is combined with unit 402 in practice. Module 409 is the DSP processor with signal reconstruction and beamforming implementations.

The 2D ROI, the transducer array, and the point-like targets are shown in FIG. 5, by way of example. The methods presented in this embodiment can be used with 3D ROI and 3D transducers.

In addition to ultrasound, non-limiting examples of other applications that embodiments of the invention can apply are microwave imaging for breast cancer screening as well as functional brain imaging.

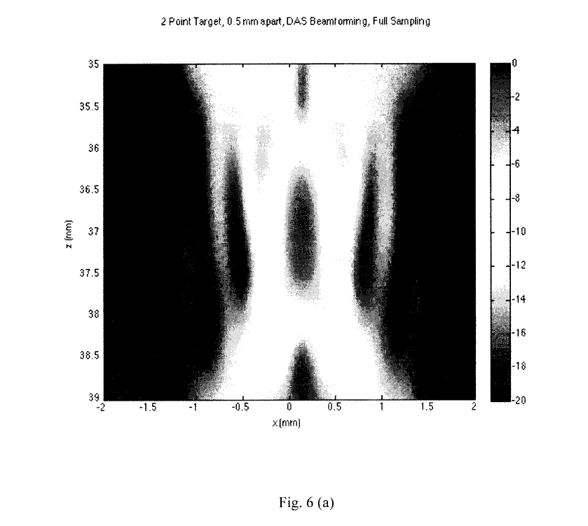

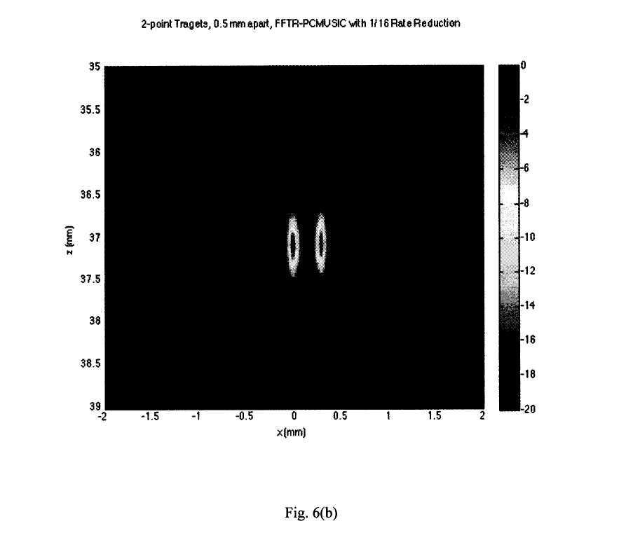

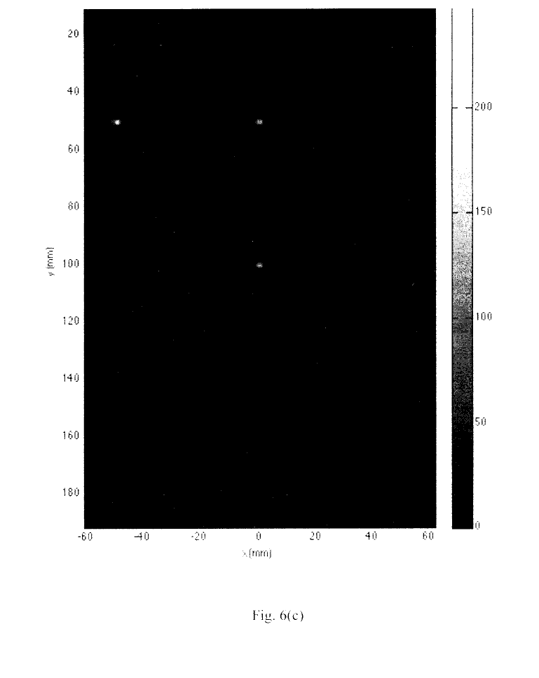

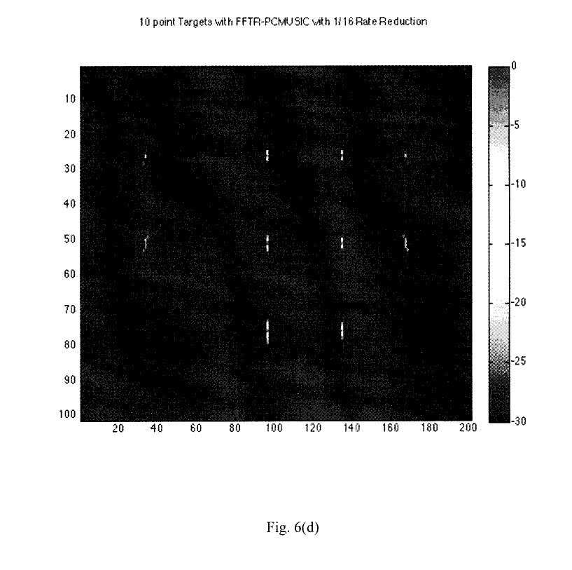

By way of example, the results from simulation of the ROI with 2, 3, and 10 point targets, real acquired data from wire phantom and the ultrasound system are demonstrated in FIGS. 6, 7, and 8. FIG. 6 (a) shows the result of simulation of two-point targets 0.5 mm apart, with full data rate and applying the DAS beamforming for the sake of comparison. FIG. 6 (b) shows the same result with 1/16 rate reduction from the low-dimensional sampling as well as applying the FFTR-PCMUSIC method. The two targets can clearly be resolved and differentiated with the method presented in this invention. FIG. 6)(c) and (d) show the results of applying same method as presented in some embodiments of the current invention to 3 and 10 point scatterers.

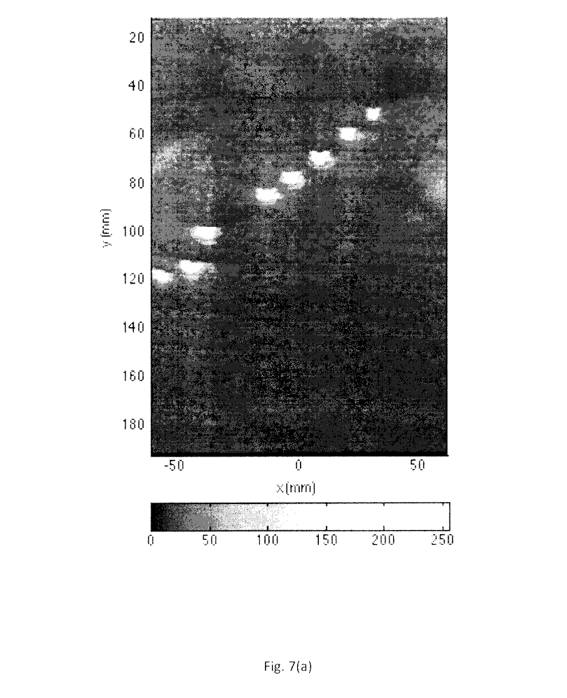

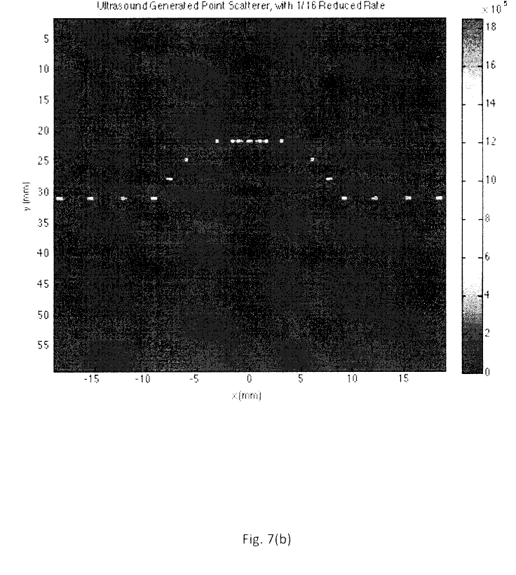

By way of example, the generated image from real ultrasound machine to a wire and point like phantom are presented in FIGS. 7 (a) and (b). Theses results are with 1/16 rate reduction and applying FFTR-PCMUSIC as the beamforming method to the data signals.

According to disclosed examples, the present disclosure provides a method including the steps of acquiring and processing ultrasound data by transmitting an ultrasound plane wave through elements of a transducer array to a Region-Of-Interest (ROI) that contains at least one point target; acquiring the signal data in response to the ultrasound data using a low-dimensional data acquisition system; reconstructing the signal data from the low-dimensional data acquisition system to a full capture data in frequency domain using compressive sensing and sparse signal recovery techniques; beamforming the full capture data with a super-resolution focused frequency technique to generate an image of the target using a time reversal matrix at the focused frequency and a green's function of the background medium at the focused frequency; and sending the image to be displayed on a display screen of an ultrasound system.

The method may be carried out using a non-transitory computer-readable medium.

The ultrasound data may be transmitted through multiple transducers reflecting the ultrasound data from the target using the low-dimensional data acquisition system.

The method may include recovering the signal data using a sparse signal recovery technique before beamforming.

The method may further include the steps of: filtering the signal data to suppress noise in a frequency band of interest; and down-sampling the signal data below the Nyquist rate using random sensing and Fourier matrices.

The recovering may be based on an optimization technique including applying a regularized l1-norm in frequency domain to estimate the data signals acquired by the low-dimensional acquisition system to the full capture data.

The signal data may be recovered from the low-dimensional sampling for a pair of transmit and receive transducers to the full capture data in frequency domain.

The beamforming may include filtering to place the signal data in an effective band of interest before generating the image.

The beamforming may include forming the time reversal matrix for multiple frequency bins within a bandwidth of interest.

The beamforming may include using focusing matrices to focus the time reversal matrix in frequency domain.

The focusing matrices may be configured to minimize the difference between the full capture data matrix at the focused frequency and the full capture data at frequency bins within the frequency band of interest.

The method may include applying a subspace-based technique to the full capture matrix in frequency domain.

The focused frequency may be formed using a weighted average of a plurality of transformed time reversal matrices at frequency bins and using a signal-to-noise ratio of the signal data within the frequency bin as weighting coefficients.

The beamforming may use the focused time reversal matrix and a time reversal PCMUSIC technique to focus spatially at the location of the targets within the ROI.

The green's function of the ROI at the focused frequency may be used to generate a pseudo-spectrum of the ROI in PCMUSIC. The pseudo-spectrum may include density contrast data relating to one or more point targets within said ROI. The green's function of the ROI may receive parameters selected from one or more of: the dimension of the transducer elements, the speed of sound, the geometry of the ROI, and the phase response of the transducer.

The beamforming may image the point targets irrespective of the targets being well resolved.

According to disclosed examples, the present disclosure also provides an apparatus including a transducer configured to send and acquire ultrasound data; a data acquisition module for low-dimensional sampling of signal data; a data processing unit for recovering the signal data from the low-dimensional ultrasound data to full-rate data; a two-dimensional image reconstructing unit to generate an image of the ROI; and a user interface module that links the data processing unit to a display screen for image display purposes.

The transducer may be in communicable connection to a computer to excite one or more elements of the transducer sequentially by a plane wave, and record the received signals from the ROI.

The ultrasound data may be acquired by the data acquisition module. The acquisition module may include processing circuitry using random Gaussian and Fourier matrices for sub-Nyquist sampling to acquire ultrasound data. The ultrasound data may be further processed by a programming executable in the data processing unit. The data processing unit may process the signal data acquired by the low-dimensional sampling unit to reconstruct an image of the ROI. The data processing unit may be configured to beamform the recovered signals using a focused frequency time reversal matrix. The data processing unit may be configured to reconstruct the image of the ROI using the pseudo-spectrum of TR-PCMUSIC technique. The image may be sent to a user interface module for display on the display screen.

While a number of exemplary aspects and examples have been discussed above, those of skill in the art will recognize certain modifications, permutations, additions and sub-combinations thereof.

1. A method comprising the steps of acquiring and processing ultrasound data by transmitting an ultrasound plane wave through elements of a transducer array to a Region-Of-Interest (ROI) that contains at least one point target; acquiring the signal data in response to the ultrasound data using a low-dimensional data acquisition system; reconstructing the signal data from the low-dimensional data acquisition system to a full capture data in frequency domain using compressive sensing and sparse signal recovery techniques; beamforming the full capture data with a super-resolution focused frequency technique to generate an image of the target using a time reversal matrix at the focused frequency and a green's function of the background medium at the focused frequency; and sending the image to be displayed on a display screen of an ultrasound system.

2. The method of claim 1, wherein the method is carried out using a non-transitory computer-readable medium.

3. The method of claim 1 wherein the ultrasound data is transmitted through multiple transducers reflecting the ultrasound data from the target using the low-dimensional data acquisition system.

4. The method in claim 1 further comprising recovering the signal data using a sparse signal recovery technique before beamforming.

5. The method in claim 1 further comprising the steps of: filtering the signal data to suppress noise in a frequency band of interest; and down-sampling the signal data below the Nyquist rate using random sensing and Fourier matrices.

6. The method in claim 4 wherein the recovering is based on an optimization technique comprising applying a regularized l1-norm in frequency domain to estimate the data signals acquired by the low-dimensional acquisition system to the full capture data.

7. The method in claim 6, wherein signal data is recovered from the low-dimensional sampling for a pair of transmit and receive transducers to the full capture data in frequency domain.

8. The method of claim 1, wherein the beamforming comprises filtering to place the signal data in an effective band of interest before generating the image.

9. The method of claim 1, wherein the beamforming comprises forming the time reversal matrix for multiple frequency bins within a bandwidth of interest.

10. The method in claim 9 wherein the beamforming comprises using focusing matrices to focus the time reversal matrix in frequency domain.

11. The method in claim 10, wherein the focusing matrices are configured to minimize the difference between the full capture data matrix at the focused frequency and the full capture data at frequency bins within the frequency band of interest.

12. The method in claim 11 further comprising applying a subspace-based technique to the full capture matrix in frequency domain.

13. The method in claim 1, wherein the focused frequency is formed using a weighted average of a plurality of transformed time reversal matrices at frequency bins and using a signal-to-noise ratio of the signal data within the frequency bin as weighting coefficients.

14. The method in claim 13 wherein the beamforming uses the focused time reversal matrix and a time reversal PCMUSIC technique to focus spatially at the location of the targets within the ROI.

15. The method in claim 14, wherein the green's function of the ROI at the focused frequency is used to generate a pseudo-spectrum of the ROI in PCMUSIC; and the pseudo-spectrum comprises density contrast data relating to one or more point targets within said ROI; and the green's function of the ROI receives parameters selected from one or more of: the dimension of the transducer elements, the speed of sound, the geometry of the ROI, and the phase response of the transducer.

16. The method in claim 14 wherein the beamforming images the point targets irrespective of the targets being well resolved.

17. An apparatus comprising: a transducer configured to send and acquire ultrasound data; a data acquisition module for low-dimensional sampling of signal data; a data processing unit for recovering the signal data from the low-dimensional ultrasound data to full-rate data; a two-dimensional image reconstructing unit to generate an image of the ROI; and a user interface module that links the data processing unit to a display screen for image display purposes.

18. The apparatus in claim 17, wherein the transducer is in communicable connection to a computer to excite one or more elements of the transducer sequentially by a plane wave, and record the received signals from the ROI.

19. The apparatus in claim 18 wherein the ultrasound data are acquired by the data acquisition module.

20. The apparatus in claim 19 wherein the acquisition module comprises processing circuitry using random Gaussian and Fourier matrices for sub-Nyquist sampling to acquire ultrasound data.

21. The apparatus in claim 20 wherein the ultrasound data are further processed by a programming executable in the data processing unit.

22. The apparatus in claim 21 wherein the data processing unit processes the signal data acquired by the low-dimensional sampling unit to reconstruct an image of the ROI.

23. The apparatus in claim 21 wherein the data processing unit is configured to beamform the recovered signals using a focused frequency time reversal matrix.

24. The apparatus in claim 21 wherein the data processing unit is configured to reconstruct the image of the ROI using the pseudo-spectrum of TR-PCMUSIC technique.

25. The apparatus in claim 24 wherein the image is sent to a user interface module for display on the display screen.
